(19) United States
(12) Patent Application Publication
(10) Pub. No.: US 2006/0063197 A1
(43) Pub. Date: Mar. 23, 2006

(54) Quality control and normalization methods for protein microarrays

(76) Inventors: Bart R. Anderson, Philadelphia, PA (US); David L. Hall, Portland, OR (US); Matthew C. Groll, Smithfield, UT (US); Christopher P. Bingham, Santa Rose, CA (US); James E. Thompson, Logan, UT (US); Rex S. Spendlove, Millville, UT (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 10/943,666

(22) Filed: Sep. 17, 2004

(51) Int. Cl. G01N 33/53; G06F 19/00

(52) U.S. Cl. 435/7.1; 702/19

(57) ABSTRACT

The use of fluorescent dye in printing buffer used to print spots of a protein microarray permits the presence, location and morphology of individual spots to be detected based on the fluorescent response of the spots. This information can be used to perform quality control of protein microarrays. In addition, the fluorescent response of the spots can prevent poor spots from being used in a protein assay or prevents faulty data from being accepted. The size of the spots can also be used to normalize the results obtained in a protein assay. The position of the spots can be used to establish anchors in detecting the chemiluminescent response of spots to which biological samples have been applied.

### BACKGROUND OF THE INVENTION

1. The Field of the Invention

The present invention relates to protein microarrays. More specifically, the present invention relates to methods for detecting the presence, location, and morphology of spots in protein microarrays and for normalizing variability in the response of microarray spots.

2. The Relevant Technology

Protein microarrays have become a necessary tool for gathering data in the field of proteomics. There are many problems yet to be worked out in producing protein microarrays. As with its predecessor, DNA microarrays, quality control and normalization of the protein array are critical. Methods to pinpoint the existence, position, morphology and spot volume for each plate, rather than sacrificing a limited number of plates from each production lot for assessment, are needed. Quality control methods for protein microarrays have yet to be well established, as protein microarrays are a relatively new technology.

Several well-known quality control methods for DNA microarrays could be modified for use with protein microarrays. However, each of these methods has drawbacks. Quality control methods for DNA microarrays have been described wherein a fluorescent probe binds to all nucleotides on the array, providing morphological, position and spot area data about the printing of that array. The major caveat to this approach is that the slides analyzed cannot then be used experimentally, and therefore the information is representative of the production lot, rather than giving specific information about the individual slide to be used experimentally.

An alternative method uses three different fluorescein-labeled probes to solve the issue of slide-specific data. One dye is used to probe all printed nucleotides, producing a qualitative measure of each printed spot, and the other two dyes are used for the experimental probes. To obtain data for the three different dye emission wavelengths, however, the use of expensive laser scanners with narrow bandwidths is required. Several methods exist for estimating production quality based on the spot morphology after hybridization or on spot intensity providing only an indirect estimate of production quality. The origin of defects is questionable due to the substantial biological variation in the assay.

While these methods have been suitable for DNA microarrays, they are not directly applicable for use with protein microarrays. Proteins as a group exhibit far more diverse physical and reactive properties than do nucleotides, which prevents the DNA methods from being successfully adopted for protein microarrays. Thus, there is a need for methods for achieving quality control for protein microarrays, particularly those that could be applied to individual slides as opposed to destructive tests that are applied only to sample slides in a lot.

### BRIEF SUMMARY OF THE INVENTION

Embodiments of the present invention relate to methods for performing quality control to the process of printing spots in protein microarrays. These methods are capable of detecting the presence, location and morphology of individual spots in microarray slides that are to be used to process biological samples. Rather than being capable of testing only selected slides in a lot in a destructive manner, the methods of the invention can be used to test individual microarrays that are later to be used for testing samples.

These results are achieved according to the invention by adding a fluorescent dye to the printing buffer that is used to print spots in a protein microarray. The fluorescent dye can be used to detect the area, position and morphology of the printed spots. This information can be used in a variety of ways. For example, the quality control information obtained by detecting the fluorescent response of printed spots can be used during microarray fabrication to determine whether individual microarrays or individual wells comply with manufacturing specifications. This information can also be used during testing of a biological sample in a visual control step to detect spot presence in situations in which there is an unexpected reaction response. In addition, the morphology and location of the spot can be quickly and easily verified to prevent the use of poor spots in assays, preventing the waste of reagents and time, as well as preventing the use of unreliable data.

Imaging and analysis software, which often struggle to identify spots that produce low responses, may be made to take advantage of the universal presence and similar response levels produced by the fluorescent image whether a low, medium or high response. The spot positions from the fluorescent image can also serve as anchors for measurement of the protein based assay spot responses. Finally, to improve the accuracy of data, the intensity of the fluorescent response generated by the dye can be used to obtain correction factors that normalize protein based assay results by adjusting for the volume of deposition for each spot.

These and other objects and features of the present methods will become more fully apparent from the following description and appended claims, or may be learned by the practice of the methods as set forth hereinafter.

### BRIEF DESCRIPTION OF THE DRAWINGS

To further clarify the above and other advantages and features of the present methods, a more particular description of the methods will be rendered by reference to specific embodiments thereof which are illustrated in the appended drawings. It is appreciated that these drawings depict only typical embodiments of the methods and are therefore not to be considered limiting of its scope. The methods will be described and explained with additional specificity and detail through the use of the accompanying drawings in which:

FIG. 1a illustrates a linear relationship between spot area and spot volume.

FIG. 1b illustrates a linear relationship between protein concentration and spot volume.

FIG. 1c illustrates a plate printed with nine different spots per well, each containing fluorescent dye in the printing buffer.

FIG. 1d illustrates a magnified view of a well of FIG. 1c.

FIG. 2a illustrates an image of a plate printed with fluorescent dye in the printing buffer.

FIG. 2b illustrates selected columns of a 96-well plate each spot constituting a full CEA Enzyme Linked Immunosorbent Assay (ELISA) imaged for chemiluminescence after being printed using a CAb printing buffer containing a fluorescent dye.

FIG. 2c illustrates the correlation between the protein based assay response and fluorescent response to different spot volumes.

FIG. 3a illustrates an image of a plate after printing, containing a fluorescent dye in the buffer of the first four rows of wells.

FIG. 3b illustrates the plate of 3a imaged for chemiluminescence after a full protein based microarray reaction has been performed.

### DETAILED DESCRIPTION OF THE PREFERRED EMBODIMENTS

Embodiments of the present invention relate to the use of fluorescent dyes in the printing of spots in protein microarrays. Microarrays formed in this manner can be conveniently subjected to quality control analysis and can be used to improve the efficiency and accuracy of tests of biological samples. Because of the relationship that has been observed, as described herein, between spot volume and spot area, the methods of the invention can be used to obtain information about the area or volume of individual microarray spots. The spotted volume affects both spot area and protein concentration per surface area, which ultimately leads to changes in test response per unit analyte. Thus, information obtained using the methods described herein can be used to determine the reliability of assays and to normalize testing results.

Embodiments of the methods disclosed herein are capable of yielding information about spot presence, location, and morphology and allow measurement of deposition volume for each spot printed, without interfering in assay performance or requiring the sacrifice of samples from each production lot. These methods are generally inexpensive, easy to perform in any laboratory, and are transferable between various microarray platforms.

In order to illustrate these and other aspects of the invention, the following discussion relates to observed relationships between spot volume and area and discoveries made during experiments and research performed by incorporating fluorescent dyes into printing buffers used to print spots of protein microarrays. While embodiments of the invention and specific experimental results are disclosed hereinbelow, the invention is not limited to these specific examples. Rather, the use of fluorescent dyes and other related principles of the invention are applicable to a variety of microarray fabrication processes as well as testing of samples using the resulting microarrays.

Spot Volume and Spot Area

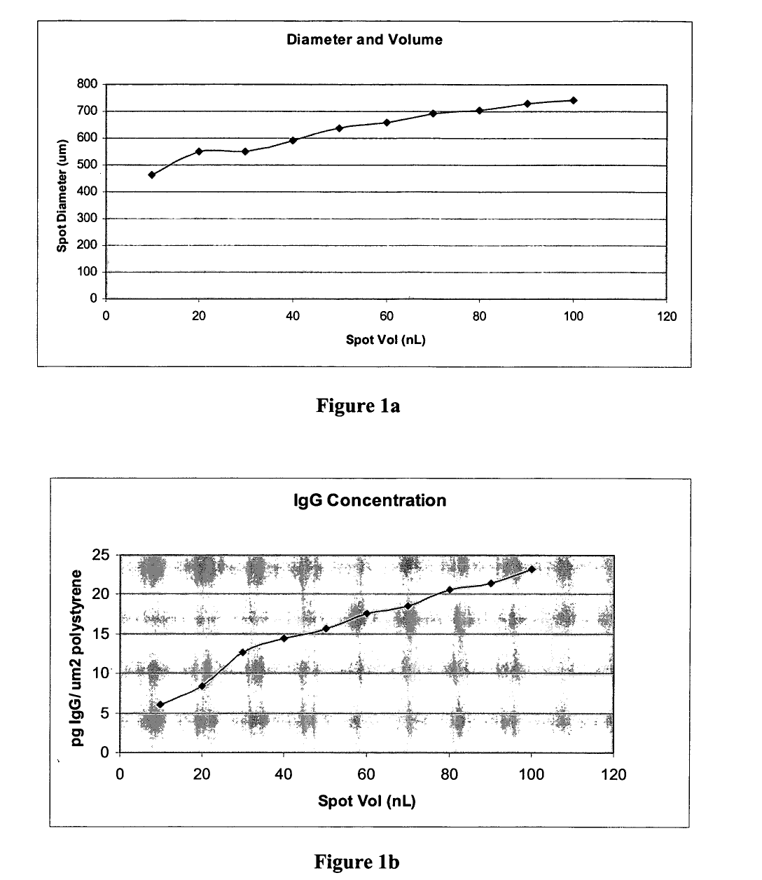

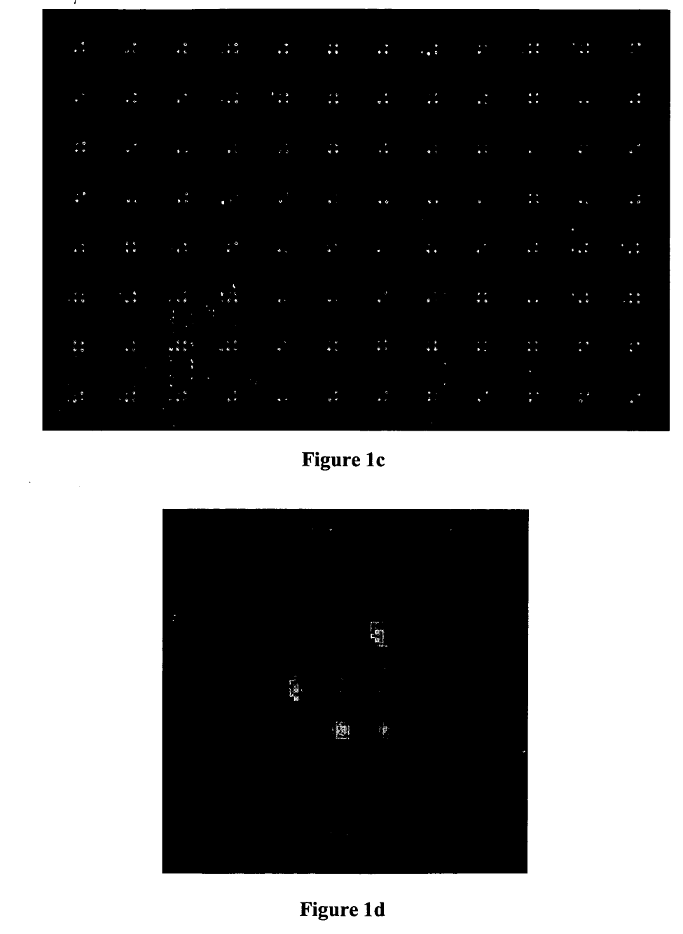

Based on experiments and research, including examples thereof disclosed herein, the inventors have discovered and observed the relationship between spot volume and spot area. In general, liquid handling robotics may be used for production of the protein microarrays, which control deposition based on volume rather than the traditional spot area. The observed linear relationship between the spot area and volume is illustrated in FIG. 1a. As the concentration of a protein in the printing buffer, deposition volume, and spot area were known, the concentration of protein per area was calculated as illustrated in FIG. 1b. As is shown in both FIGS. 1a and 1b, alteration in the printed spot volume affects the area the spot occupies and alters the concentration of protein available per unit surface area, and therefore alters the assay response.

Spots may be printed, for example, in 10 nL increments from 250 to 10 nL in successive wells of a 96-well plate. Spot printing in this manner can be achieved using a Cartesian PixSys 4500 liquid-handling robot, for example. Those of skill in the art will appreciate that the spot volumes can be larger or smaller than those presented in the foregoing example, and that the invention can be practiced using any suitable spot printing technology, including those that currently are available and those that might be developed in the future. Microarray substrates include, but are not limited to, methylcellulose, microscope slides, chips and plates, that may or may not include wells. In this example, each spot diameter is measured by placing the plate on a hemocytomer slide with 0.05 mm rulings and observing through 40× optical magnification, for example. The diameter of each spot is used to calculate the spot area. An average measurement of nine spots, for example, may be used at a spot volume of 50 nL.

Hydrophobicity and hydrophilicity of the solid phase, salt content of the printing buffer, surface contaminants and the method of deposition are direct determinants of the area a given volume will occupy. With constant printing conditions, the spot volume has a linear relationship to the spot area. Variation in spot volume causes variation in the concentration of protein available per surface area, which in turn causes variation in the amount of protein available for assay activity. This underlies the importance of being able to measure and make adjustments for variation in deposition volume.

Spot Presence, Location and Morphology

Multiple fluorescent dyes may be used to test spot presence, location and morphology, including RHODAMINE BLUE, molybdatetungstatephosphate; TEXAS RED, 1H,5H,11H,15H-Xantheno[2,3,4-ij:5,6,7-i′j′]diquinolizin-18-ium; ALEXA FLUOR 647, 8-(6-aminohexyl)aminoadenosine 3′ or 5′-cyclicmonophosphate; ALEXA FLUOR 594, Pyrano[3,2-g:5,6-g′]diquinolin-13-ium; ALEXA FLUOR 546, Benzoic acid; RHODAMINE RED, C₃₇H₄₄N₄O₁₀S₂; TETRAMETHYLRHODAMINE, Xanthylium; and MARINA BLUE (MB), 2,5-Pyrrolidinedione. With the proper excitation and filter equipment, each dye can be used according to embodiments of the invention. A preferred method of adding fluorescent dye involves the use of an unconjugated fluorescent dye. Other techniques can be used, including adding a fluorescent dye conjugated to a streptavidin molecule, adding a fluorescent dye conjugated to IgG, and directly conjugating a fluorescent dye to an antibody.

In one example, which is illustrated in FIGS. 1c and 1d, unconjugated MB molecule (6,8-difluoro-7-hydroxy-4-methylcoumarin, sold by Molecular Probes of Eugene, Oreg.) was added into the printing buffer. This method had the benefits of reduced cost and time investment relative to the various conjugation methods. A digital picture was taken to allow the location and shape of the printed spots to be visually assessed, or opened in specific software packages for more detailed statistical analysis. This may be accomplished by producing a common image file that can be opened with any number of PC or Mac software programs. The coefficient of variation (CV) of responses with and without normalization was compared to demonstrate the effect of normalization on data reproducibility. The spot area was graphed as a function of spot volume to establish the relationship between the two figures. The area per volume varies with spotting conditions, but for a constant surface material and deposition method, there remains a constant relationship between the two.

In an alternative example, MB conjugated to streptavidin was added to the printing buffer, and although it produced a quantifiable response, the ELISA reaction was depressed by the addition. In another example, the same effect was seen when MB conjugated to an IgG was added. The ELISA depression is presumably a result of competition for solid phase binding between the capture antibody (Cab) molecule and the strep-MB or IgG-MB. In yet another example, attempts to conjugate the MB molecule directly to CAb resulted in an inconsistent relationship between spot volume and fluorescent response, as well as a depression of ELISA response. If direct conjugation to CAb is desired, however, these effects are mitigated by optimization of the conjugation protocol and the MB:CAb molar ratio.

FIG. 1c shows a plate printed with nine different spots, each with MB in the printing buffer. The spots are readily identifiable. A plate with several printing errors was intentionally chosen, and FIG. 1d shows a close view of one such spot, demonstrating that MB allows identification of spots with poor morphology. As illustrated in FIGS. 1c and 1d, producing an image by photographing a plate allows for rapid visual confirmation of spot quality, evidence of spot existence, verification of proper spot location and identification of spot morphology. Either by visual assessment or various software programs, spots can be precluded from use if they are absent, misplaced or irregularly shaped. This prevents waste of valuable time and resources on spots that, due to poor printing, would not give accurate or reproducible data.

Correlation to Spot Volume

A single spot of anti-carcinoembryonic antigen (CEA) antibody was printed in each well of 96-well plates with spot volumes decreasing in 10 nL increments from 100 to 10 nL, running left to right. The average fluorescent response intensity of the spots from each column was compared to the average chemiluminescent response.

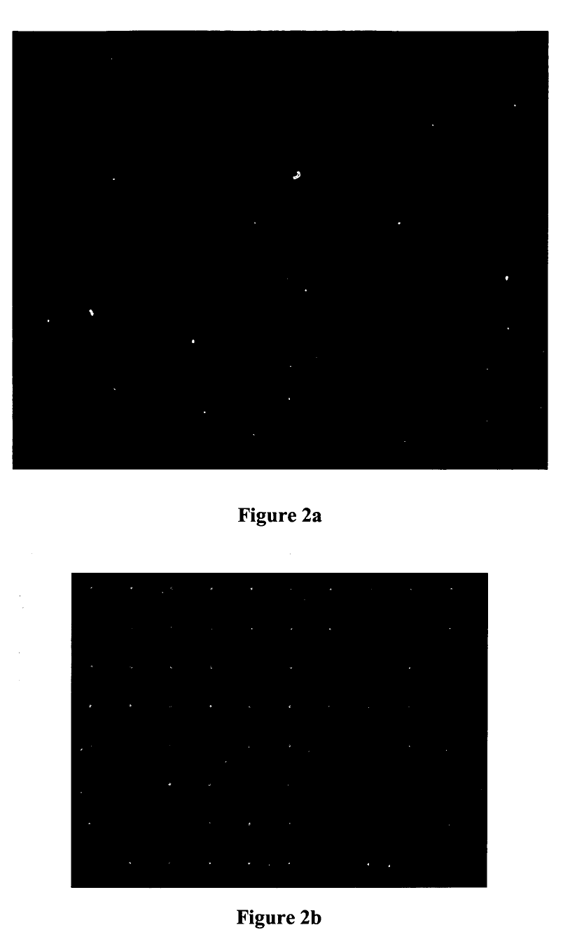

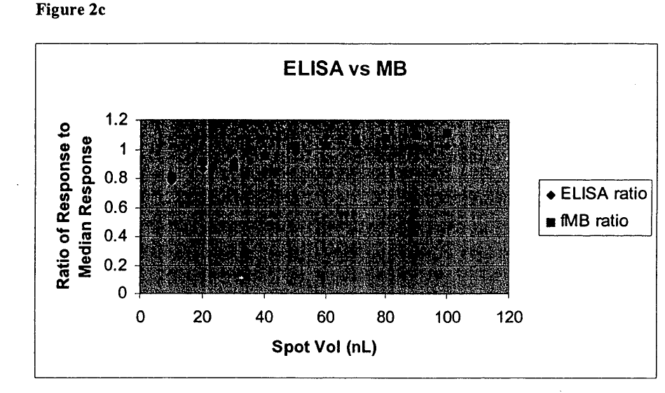

The CEA CAb was printed in a capture buffer, which included a dilution of MB. After incubation, the microarray plate fluorescence was imaged, as illustrated in FIG. 2a, using ultraviolet transillumination and a commercially available charge-coupled device (CCD) imager, which has a 500 nm bandpass filter.

An immunoassay block buffer, for example Scytek AAA SuperBlock reagent, was then added to the plates, incubated and decanted off. An antigen (Ag) concentration of 3.02 ng/mL CEA was added to the plate. A monoclonal anti-CEA detection antibody (DAb) completed the sandwich ELISA. Chemiluminescent substrate was applied, and the CCD was used for imaging without transillumination or a filter. FIG. 2b illustrates the image of selected columns after the addition of a chemiluminescent substrate.

Spot intensity responses were measured for both images using image analysis software developed by the Spendlove Research Foundation. For both fluorescent and chemiluminescent results, the median response value was found and the ratio of the median to the individual spot was calculated. The average of this proportional response was taken for each deposition volume, and the MB proportional response and ELISA proportional response were graphed together (see FIG. 2c). For comparison, each point was graphed as the ratio of the spot response to the median response. Summary statistics were calculated for the relationship between the Marina Blue and ELISA responses using GraphPad Prism Version 4.00 for Windows, GraphPad Software, San Diego Calif. USA. FIG. 2c illustrates that a linear relationship is seen between 10 nL and 70 nL, after which the response plateaus for both the MB and ELISA response. To show co-linearity, the spot response was divided by the median response to give a proportional response.

Finally, to statistically support the correlation between the MB and ELISA responses throughout the range of linearity, the paired responses were statistically evaluated, showing a strong Pearson correlation and a two-tailed p-value of 0.0002 (see Table 1). As shown, the correlation is strong from 20 nL through 70 nL, after which the ELISA response is lower in proportion to the fluorescent response, preventing use of fluorescent response for normalization outside the range of 20 nL through 70 nL deposition volume. It is thought that a saturation response occurs and, by altering the concentration of MB included and the image exposure time, a linear response is possible through the range of larger volumes if so desired.

TABLE 1

| Correlation | |
|---|---|
| Number of XY Pairs | 79 |
| Pearson r | 0.4137 |
| 95% confidence interval | 0.2120 to 0.5817 |
| P value (two-tailed) | 0.0002 |
| P value summary | *** |
| Is the correlation significant? | Yes |
| (alpha = 0.05) | |
| R squared | 0.1712 |

Effect on ELISA

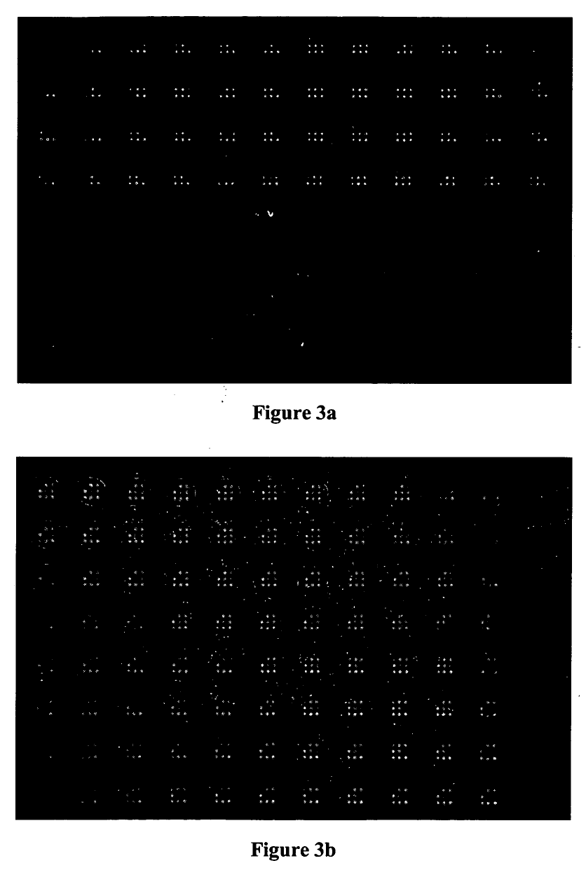

Microarrays containing nine analytes per well were printed in 96 well plates. A capture antibody buffer containing MB was printed on half of the plate, and on the other half of the plate the same CAb and buffer without MB was printed. To address the concern that the addition of MB may increase the variability of the ELISA response, and therefore decrease the reproducibility, the CV of ELISA response was measured with and without the addition of MB in the printing buffer. After incubation, the plates were imaged using fluorescent excitation, as illustrated in FIG. 3a, and blocking solution was then added to the plates as previously described. An antigen cocktail composed of known analyte levels was added to all wells, where each of the nine spots in a well contains Cab for a different Ag. After incubation, the plates were washed with Tris-buffered saline solution with Tween®-20 (TBST) three times using a BioTek Elx405 automated microplate washer, for example. A DAb cocktail was added to the plate, incubated, and the plates were again washed, this time six times with TBST. Substrate was added and the plate was imaged for chemiluminescence after a fill ELISA reaction, as illustrated in FIG. 3b.

Average response for each analyte was calculated separately, with separate averages calculated for the spots printed with and without MB. The CV of responses for each analyte system was also calculated separately for the replicates with and without MB. Three replicate plates were tested, and the average of the three plates was calculated as well. The intensity responses and CV for each system, as well as the nine-system average, were compared for spots with and without MB.

Table 2 shows the effect on chemiluminescent response as well as the effect on response CV between wells for each of the nine antigens. The effect on the ELISA response appears to vary to some degree. For example, adding MB to the spotting buffer consistently had a slightly negative effect on the response of the Alpha-Fetoprotein (AFP) ELISA, but also consistently had a slightly positive effect on the ELISA for Follicle Stimulating Hormone (FSH). On average, there was a 6.71% decrease in response intensity with MB added to the printing buffer. The response CV between wells decreased by 0.29% with MB added to the printing buffer.

TABLE 2
Effect of MB on ELISA

| Analyte | Response % Decrease | CV Increase |
|---|---|---|
| AFP | 31.68% | 3.23% |
| hCGb | −6.12% | 2.06% |
| CEA | 16.14% | −0.71% |
| PSA | −3.32% | −1.50% |
| LH | 1.11% | −2.13% |
| FSH | −43.84% | −0.85% |
| 15-3 | 19.39% | 0.52% |
| 19-9 | 31.50% | −1.63% |
| 125 | 13.81% | −1.58% |
| Avg | 6.71% | −0.29% |

There was a small decrease in CV with the addition of MB, indicating MB does not decrease the reproducibility of the assay. It is thought that the small size of MB allows it to be in solution without significant interference, however, the small decrease in ELISA response may be due to the physical presence of MB sterically blocking the binding of the CAb to the solid phase. The MB used was not activated for binding to the plate or to the proteins, and so it would be expected to wash away with other non-binding buffer components. However, when fluorescently imaged after ELISA (including washing steps) but prior to addition of substrate, spots were still slightly visible, indicating some residual MB, likely due to non-specific binding to the solid phase or CAb.

Normalization

Microarrays containing nine analytes per well were printed in 96 well plates. The plates were imaged for MB response as previously discussed. An ELISA was then performed on each plate. A standard curve was run in duplicate, the remainder of the plate being dedicated to samples with known levels for each of the nine analytes. Samples at the high end, low end and middle of the standard curve range were tested to avoid biasing results toward a specific analyte concentration. Both the fluorescent MB and chemiluminescent ELISA images were analyzed for each spot response. The median fluorescent response was calculated separately for each analyte. The ratio of median fluorescent response to individual spot fluorescent response was calculated for each spot. Each spot was normalized by multiplying the chemiluminescent response of the spot by its individual fluorescent ratio.

The average effect of normalization on CV over two plates at three different Ag concentrations is shown in Table 3. In each case the CV improves. The average CV over all three concentration levels on two plates shows a CV improvement of 1.97%.

TABLE 3

| 2 Plate Avg CV Improvement: | 1.97% |
|---|---|
| Low Ag Conc (1:50) CV Impr. | 1.74% |
| Med Ag conc (1:10) CV Impr. | 1.84% |
| High Ag Conc (1:2) CV Impr. | 2.32% |

Normalization through multiplying each spot's ELISA response by the ratio of the median fluorescence to the spot's fluorescence was shown to improve data repeatability, as indicated in Table 3. A reduction in CV for replicate testing of the same sample was shown when a spot-specific normalization for spot volume was applied. Testing at high, medium and low concentrations, covering the range of the standard curve, shows that this normalization improves results regardless of analyte concentration. The effect is proportional to spot volume, not a function of analyte concentration or biased toward a specific chemiluminescent intensity. The amount of Ag that can be detected is a function of the functional CAb bound to the solid phase. As shown in FIG. 1b, the CAb spot volume is generally proportional to the ELISA response.

A single source is used to supply the CAb for all the spots on any given plate, so the concentration is unchanged. Variation in the solid phase has not been observed. However, given the relationship between spot volume and area, as well as the fact that the chemistry of protein binding is altered on a variable solid phase, using a consistent solid phase surface is important in this embodiment of the invention. Under constant CAb concentration and a consistent solid phase surface, spot volume can be measured through the processes previously noted. ELISA response has been shown herein to vary in proportion to spot volume, and that relationship is predicted as a result of the CAb available for binding to the solid phase.

### CONCLUSION

Incorporating a fluorescent dye into protein microarray production allows each spot to be individually quality assured, with a negligible effect on the protein assay. The process removes the necessity of removing some plates from each production lot for quality assessment and quality control purposes. Additionally, this method eliminates the unavoidable doubt that remains when one assumes that the plates assayed for quality assessment and quality control from a lot are truly representative of every assay in the lot. The materials and time commitment required to add this process to array production are minimal.

A fluorescent image provides a quick visual control of spot presence in cases where reaction shows an unexpected response. The morphology and location of the spot can be quickly and easily verified to prevent the use of poor spots in assays, preventing the waste of reagents and time, as well as preventing the use of unreliable data. The ratio of spot response to median response is proportional to the changes in spot volume and as such can be used as a measure of variation in the quality assessment and quality control of individual plates or production processes.

Imaging and analysis software, which often struggle to identify spots that produce low responses, may be made to take advantage of the universal presence and similar response levels produced by the fluorescent image. The spot positions from the fluorescent image may serve as anchors for measurement of the protein based assay spot responses, which vary dramatically in existence and intensity with concentration of the sample being tested. Finally, to improve the accuracy of data, the intensity of the fluorescent response generated by the dye can be used to normalize protein based assay results by adjusting for the volume of deposition for each spot.

The present methods may be embodied in other specific forms without departing from its spirit or essential characteristics. The described embodiments are to be considered in all respects only as illustrative and not restrictive. The scope of the methods is, therefore, indicated by the appended claims rather than by the foregoing description. All changes which come within the meaning and range of equivalency of the claims are to be embraced within their scope.

What is claimed is:

1. A method for performing quality control of printed protein microarrays, comprising:
obtaining a printing buffer that includes a fluorescent dye and one or more analytes;
using the printing buffer to print spots in regions of a protein microarray; and
detecting a fluorescent response of the printed spots, thereby detecting at least one of spot presence, spot location and spot morphology.

2. The method of claim 1, further comprising using the microarray in a protein assay.

3. The method of claim 1, further comprising:
identifying regions on an array that have spots having characteristics that permit the regions to be designated for use in a protein assay; and
identifying other regions that have characteristics such that the regions are not to be used in a protein assay.

4. The method of claim 1, further comprising storing information associated with said at least one of spot presence, spot location and spot morphology, such that said stored information can be later used during a protein assay to enable results of the protein assay to be interpreted in view of said information.

5. The method of claim 1, further comprising storing information associated with said at least one of spot presence, spot location and spot morphology, such that said stored information can be later used to permit specific regions of the microarray to be used in a protein assay.

6. The method of claim 1, further comprising measuring a volume of spots of the microarray based on a detected area of the spots and a known relationship between area and volume.

7. The method of claim 1, wherein the fluorescent dye is selected from the group consisting of:
molybdatetungstatephosphate;
1H,5H, 11H,15H-Xantheno[2,3,4-ij:5,6,7-i′j′]diquinolizin-18-ium;
8-(6-aminohexyl)aminoadenosine 3′;
5′-cyclicmonophosphate;
Pyrano[3,2-g:5,6-g′]diquinolin-13-ium;
Benzoic acid;
Rhodamine Red;
Xanthylium; and
2,5-Pyrrolidinedione.

8. The method of claim 1, wherein detecting a fluorescent response of the printed spots comprises obtaining a digital image of the microarray under conditions that permit the fluorescent dye to fluoresce.

9. The method of claim 1, further comprising:
discarding the microarray if it is determined that the detected fluorescent response indicates that the printed spots are unacceptable for use; and
accepting the microarray if it is determined that the detected fluorescent response indicates that the printed spots are acceptable for use.

10. The method of claim 1, wherein the microarray is formed on a plate.

11. The method of claim 10, wherein the plate comprises a microscope slide.

12. The method of claim 1, wherein the microarray is formed on a methylcellulose substrate.

13. The method of claim 1, wherein the microarray comprises a chip.

14. The method of claim 1, wherein the microarray includes wells.

15. A method of performing a protein assay using a protein microarray, comprising:
obtaining a protein microarray that includes regions having spots that incorporate a fluorescent dye;
applying a biological sample to selected regions of the protein microarray; and
based on information associated with a fluorescent response of the spots of the protein microarray, performing at least one of:
selection of the regions to which the biological sample is applied;
determining whether to accept data obtained from spots of the regions to which the biological sample has been applied; and
locating the spots to be analyzed.

16. The method of claim 15, further comprising performing selection of the regions to which the biological sample is applied, wherein the fluorescent response of the spots of the protein microarray identifies regions that do not have spots suitable to be used in the protein assay.

17. The method of claim 15, further comprising determining whether to accept the data, wherein the fluorescent response of the spots of the protein microarray identifies regions that do not have spots that are capable of yielding acceptable data.

18. The method of claim 15, further comprising determining whether to accept the data, including:
determining that a particular region has yielded unexpected results; and
using the fluorescent response to determine whether the particular region has spots that are capable of yielding acceptable data.

19. The method of claim 15, wherein obtaining the protein microarray comprises obtaining from the protein microarray manufacturer information associated with the fluorescent response of spots of the protein microarray, wherein the information associated with the fluorescent response has been mapped to individual spots of the microarray.

20. The method of claim 15, further comprising, prior to applying the biological sample, detecting the fluorescent response so as to generate the information associated with a fluorescent response.

21. The method of claim 15, wherein the information associated with the fluorescent response relates to at least one the presence, location and morphology of the spots.

22. A method of normalizing results obtained in a protein assay performed using a protein microarray, comprising:
obtaining a protein microarray that includes regions having spots that incorporate a fluorescent dye;
applying a biological sample to selected regions of the protein microarray; and
based on information associated with a fluorescent response of at least one of the spots:
calculating a correction factor associated with a particular spot; and
adjusting a value associated with the quantitative reaction between an analyte of the particular spot and the biological sample by the corrective factor to obtain a normalized result for the particular spot.

23. The method of claim 22, wherein the correction factor is proportional to a spot volume of the particular spot that has been measured by detecting the fluorescent response of the particular spot.

24. The method of claim 22, wherein the correction factor is obtained by calculating a ratio of an average fluorescent response of the spots of the microarray to the fluorescent response of the particular spot.

25. The method of claim 22, wherein:
the protein assay is an enzyme linked immunosorbent assay;
the value is proportional to a chemiluminescent response of the particular spot; and
adjusting the value comprises multiplying the value by the correction factor.

26. A method of performing an enzyme linked immunosorbent assay using a protein microarray, comprising:
obtaining a protein microarray that includes regions having spots that incorporate a fluorescent dye;
obtaining information specifying a location of at least one of the spots that has been generated by detecting a fluorescent response of said at least one of the spots;
using the information specifying the location of at least one of the spots to establish an anchor for detecting the chemiluminescent/fluorescent response of other spots of the protein microarray.
